(19) United States
(12) Patent Application Publication
(10) Pub. No.: US 2026/0165730 A1
(43) Pub. Date: Jun. 18, 2026

(54) THROMBECTOMY SYSTEM AND METHOD OF REMOVING THROMBUS

(71) Applicant: Shifamed Holdings, LLC, Campbell, CA (US)

(72) Inventors: Aadel AL-JADDA, San Carlos, CA (US); Muralidharan SRIVATHSA, Santa Clara, CA (US); Paul GUNNING, Montara, CA (US); Uday ILLINDALA, Los Gatos, CA (US)

(21) Appl. No.: 19/123,734

(22) PCT Filed: Oct. 31, 2023
(86) PCT No.: PCT/US2023/078287

§ 371 Date: Apr. 23, 2025

Related U.S. Application Data
(60) Provisional application No. 63/381,725, filed on Oct. 31, 2022.

(51) Int. Cl. A61B 17/3207; A61B 17/22; A61B 17/221; A61B 17/34; A61F 2/01

(52) U.S. Cl. CPC A61B 17/32075; A61B 17/22; A61B 17/221; A61B 17/320725; A61B 17/3478; A61F 2/013

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus. The system can include mechanical engagement features for manipulating and/or urging clot material into an aspiration lumen of the catheter.

### PRIORITY CLAIM

This patent application claims priority to U.S. provisional patent application no. 63/381,725, titled “THROMBECTOMY SYSTEM AND METHOD OF REMOVING THROMBUS,” and filed on Oct. 31, 2022, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to thrombectomy systems for removing thrombus, and in various aspects to systems including aspiration and associated methods for removing a thrombus from a mammalian blood vessel.

### BACKGROUND

Thrombotic material may lead to a blockage in fluid flow within the vasculature of a mammal, such as a human. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Embolic events have historically been treated with slow-acting pharmacologic agents such as lytic therapy or catheter-directed therapy. Clinicians have turned to mechanical thrombectomy increasingly in recent years with the advent of more effective therapies. Examples of mechanical thrombectomy systems include aspiration catheters designed to enable easier navigation to the clot for removal and produce larger aspiration forces.

Existing mechanical thrombectomy systems suffer from several drawbacks, however. Systems relying on aspiration alone require larger catheters for larger clot. Softer clot may potentially be removed through a smaller profile catheter, but the morphology of clots varies and cannot be determined in advance. Indeed, clinicians oftentimes can only assess the clot after it has been removed from the body. Likewise, larger catheters cannot access smaller vessels. For this reason, clinicians must make a tradeoff between aspiration on the one hand and navigation and access on the other hand.

Even larger catheters struggle to remove organized clot because it cannot be sucked down into the relatively smaller aspiration lumen. In many cases the clinician captures the clot in the distal end of the catheter with aspiration (sometimes called “lollipopping”) and removes the catheter with the thrombus. This requires multiple introductions of the catheter in the patient which introduces significant risk of complications and adds precious time to the emergent procedure.

Other tools have been developed to remove difficult clot like clot retrievers, but these tools are not being widely adopted because of their limited effectiveness and additional costs versus aspiration or the standard of case.

For the above and other reasons, many patients presenting with deep vein thrombus (DVT) are left untreated as long as the risk of limb ischemia is low. There remains the need for a device to address these and other problems with existing thrombectomy systems including, but not limited to, a fast, easy-to-use, and effective device for removing a variety of clot morphologies. There remains the need for a device for removing a variety of clot morphologies in smaller vessels and deeper in the peripheral vasculature. There remains the need for a device for removing a variety of clot morphologies in a single pass.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1 and 1A illustrate various views of a distal portion of a thrombus removal system including a funnel in accordance with various embodiments of the present technology.

FIGS. 2A-2E illustrate plan views of various configurations of irrigation ports and fluid streams for use with the catheter of FIG. 1A.

FIGS. 3A-3E illustrate an elevation view of various configurations of irrigation ports and fluid streams for use with the catheter of FIG. 1A.

FIGS. 4A and 4B illustrate an exemplary funnel assembly for use in connection with the system described herein.

FIGS. 5A and 5B illustrate another exemplary funnel assembly for use in connection with the system described herein.

FIG. 6 is a flowchart describing a method of removing a thrombus from a patient.

FIGS. 7A, 7B, 8A, and 8B show an exemplary catheter as described above with an expandable funnel in fluid jets.

FIGS. 9A-9F is a sequence of figures showing a similar catheter but including ribs for manipulating clot material inside the funnel.

### SUMMARY OF THE DISCLOSURE

A thrombus removal system is provided, comprising mechanisms to capture and remove clot from the body. In various embodiments, the system includes mechanically actuated arms for pulling clot into the aspiration lumen. In various embodiments, the mechanically actuated arms are formed within an expandable distal end of the catheter. In various embodiments, the expandable distal end of the catheter is formed as a funnel. In various embodiments, the catheter includes ports for delivering fluid jets to disrupt or break up the receiving clot such that the clot material can be aspirated through a smaller lumen.

A thrombus removal device is provided, comprising: an elongate catheter having an aspiration lumen; an aspiration source coupled to the aspiration lumen; an expandable funnel coupled to the aspiration lumen and the elongate catheter; at least one mechanical engagement feature disposed entirely external to the expandable funnel, wherein the at least one mechanical engagement feature is actuatable to pass through an opening in expandable funnel into an interior of the expandable funnel.

In some aspects, the at least one mechanical engagement feature is actuatable to move the at least one mechanical engagement towards the aspiration lumen.

In one embodiment, the at least one mechanical engagement feature is actuatable to move the at least one mechanical engagement feature generally radially within the expandable funnel across at least a portion of the expandable funnel.

In another aspect, the at least one mechanical engagement feature comprises a cutting portion.

In some aspects, the at least one mechanical engagement feature comprises a blunted tip.

In another aspect, the device includes a fluid source; a fluid lumen positioned in the elongate catheter and in fluid communication with the fluid source; a jet orifice positioned near or within the expandable funnel and in fluid communication with the fluid lumen, the jet orifice being configured to provide a fluid stream within the aspiration lumen or within the expandable funnel.

In some aspects, the at least one mechanical engagement feature is actuatable to move the at least one mechanical engagement within the expandable funnel towards the fluid stream.

In one aspect, at least one mechanical engagement feature is in fluid communication with the fluid lumen and is further configured to provide a second fluid stream within the aspiration lumen or within the expandable funnel.

In some aspects, the expandable funnel comprises a funnel frame configured to self-expand the funnel to a fully expanded configuration.

In some aspects, the expandable funnel further comprises a compliant material disposed over at least a portion of the funnel frame.

In one aspect, the at least one mechanical engagement feature includes an at-rest configuration in which the at least one mechanical engagement feature is positioned adjacent to or abutting against an exterior surface of the compliant material.

In some aspects, the compliant material further comprises at least one opening corresponding to each of the at least one mechanical engagement features, wherein the at least one mechanical engagement is configured to pass through the at least one opening in the compliant material when actuated.

In another aspect, actuation of the at least one mechanical engagement feature causes the at least one mechanical engagement feature to pivot within the expandable funnel.

In some aspects, the at least one mechanical feature does not extend beyond a distal end of the expandable funnel.

In one aspect, at least a distal end of the at least one mechanical engagement feature is actuatable to pass through the opening in expandable funnel into the interior of the expandable funnel.

A medical device is provided, comprising: an elongate catheter; an expandable member positioned at a distal end of the elongate catheter; at least one mechanical engagement feature disposed exterior to the expandable member, wherein at least a distal end of the at least one mechanical engagement feature is actuatable to pass through an opening in the expandable member into an interior of the expandable member.

In some aspects, the at least one mechanical engagement feature is actuatable to move the at least one mechanical engagement within the expandable member.

In another aspect, the at least one mechanical engagement feature is actuatable to move the at least one mechanical engagement feature generally radially within the expandable member across at least a portion of the expandable member.

In some aspects, the at least one mechanical engagement feature comprises a cutting portion.

In another aspect, the at least one mechanical engagement feature comprises a blunted tip.

In some aspects, the device further includes: a fluid source; a fluid lumen positioned in the elongate catheter and in fluid communication with the fluid source; a jet orifice positioned near or within the expandable member and in fluid communication with the fluid lumen, the jet orifice being configured to provide a fluid stream within the expandable member.

In some aspects, the at least one mechanical engagement feature is actuatable to move the at least one mechanical engagement within the expandable member towards the fluid stream.

In other aspects, at least one mechanical engagement feature is in fluid communication with the fluid lumen and is further configured to provide a second fluid stream within the aspiration lumen or within the expandable member.

In one aspect, the expandable member comprises a frame configured to self-expand the expandable member to a fully expanded configuration.

In some aspects, the expandable member further comprises a compliant material disposed over at least a portion of the frame.

A method of removing thrombus from a patient, comprising: inserting a thrombectomy catheter into the patient; expanding a distal expandable member of the catheter adjacent to a target thrombus; aspirating the target thrombus into the distal expandable member; actuating at least one mechanical engagement feature to pass from an exterior of the expandable member to be within the expandable member to contact the target thrombus; and aspirating the target thrombus out of the thrombectomy catheter.

In some aspects, the method includes directing at least two intersecting jet streams into the target thrombus within the distal expandable member.

In one aspect, actuating the at least one mechanical engagement feature further comprises cutting the target thrombus with the at least one mechanical engagement feature In some aspects, actuating the at least one mechanical engagement feature further comprises pinching the target thrombus with the at least one mechanical engagement feature.

In other aspects, actuating the at least one mechanical engagement feature further comprises shearing the target thrombus with the at least one mechanical engagement feature.

In one aspect, actuating the at least one mechanical engagement feature further comprises moving the at least one engagement feature towards an aspiration lumen of the thrombectomy catheter.

In another aspect, actuating the at least one mechanical engagement feature further comprises moving the at least one engagement feature radially across the distal expandable member.

In some aspects, actuating the at least one mechanical engagement feature further comprises moving the at least one engagement feature towards a plane of the intersecting jet streams.

In some aspects, a thrombectomy catheter is provided, comprising: an aspiration lumen within an elongate catheter for removing clot from the body; a distal end of the catheter comprising a rib structure includes at least one actuatable rib; wherein the rib structure is configured such that the at least one rib moves towards the aspiration lumen.

In some aspects, the catheter includes an expandable funnel at the distal end.

In one aspect, the at least one rib is configured to be flush with the walls of the funnel the funnel is expanded and the at least one rib is unactuated, and the rib is extended away from the funnel wall when the rib is actuated.

In some aspects, axial movement of the adjustable at least one rib causes the expanded funnel shape to be modified.

In another aspect, the at least one rib and the funnel are integrated in a unitary structure.

### DETAILED DESCRIPTION

This application is related to disclosure in International Application No. PCT/US2021/020915, filed Mar. 4, 2021 (the '915 application), International Application No. PCT/US2022/033024, filed Jun. 10, 2022 (the '024 application), provisional application no. 63/381,467, filed Oct. 28, 2022, provisional no. 63/381,019, filed Oct. 26, 2022, and provisional no. 63/373,413, filed Aug. 24, 2022, the entire disclosures of which are incorporated by reference herein for all purposes. These applications describe general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

Any headings provided herein are for convenience only and do not interpret the scope or meaning of the claimed present technology.

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments through a small lumen of the catheter out of the patient's body. In examples including pressurized fluid streams (e.g., jets), the fluid streams may disrupt, cut, or macerate thrombus. Fragmentation helps to prevent clogging of the aspiration lumen and/or allows the thrombus removal system to macerate large, firm clots that otherwise could not be aspirated. As used herein, “thrombus”, “embolism”, and “clot” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

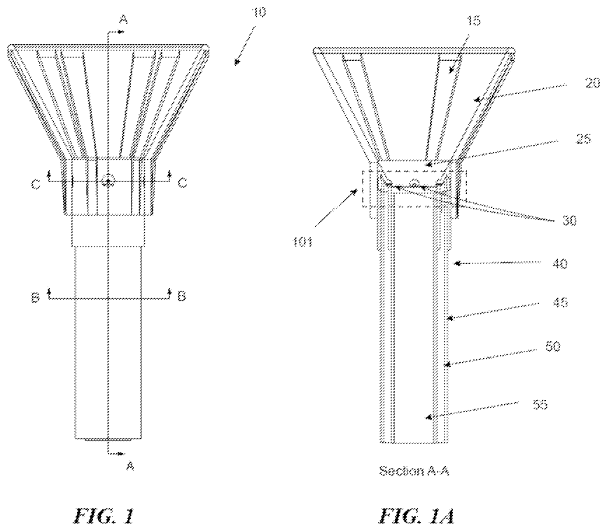

FIG. 1 illustrates a distal portion 10 of an exemplary thrombus removal system according to an embodiment of the present technology. FIG. 1A Section A-A illustrates an elevation sectional view of the distal portion. The example section A-A in FIG. 1A depicts an exemplary funnel 20 that is positioned at the distal end of the distal portion 10, the funnel adapted to engage with thrombus and/or a tissue (e.g., vessel) wall to aid in thrombus fragmentation and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

The dimensions and configurations of the system may depend on the application. Conventional thrombectomy systems are sized based on the target location. For example, a larger bore aspiration catheter can remove a higher clot burden but may be too large to access anything other than main vessels. Conversely, smaller aspiration catheters may be required for small vessels, such as the small vessels past the pulmonary trunk or peripheral vasculature and deep veins. The system described herein allows for different configurations to avoid common tradeoffs in conventional systems.

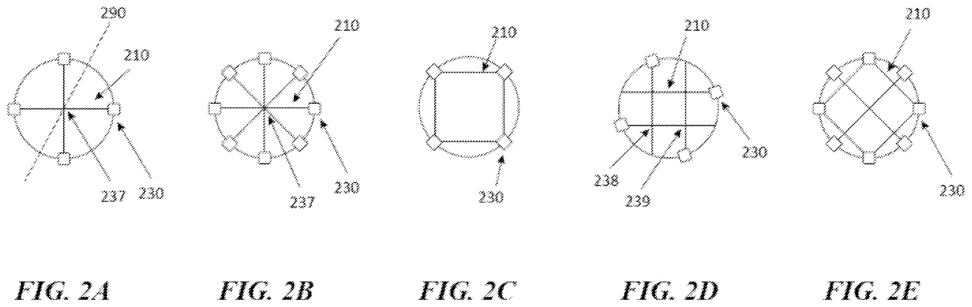

Turning to FIGS. 2A to 2E, in some embodiments, port(s) 230 is formed near the opening of the aspiration lumen to direct the fluid flow along a selected path. In the exemplary embodiment, the catheter includes a funnel and the ports are formed at the base of the funnel where clot is pulled into the aspiration lumen.

FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

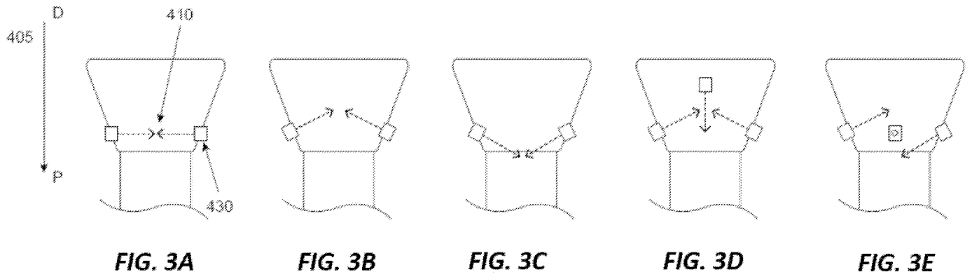

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle a may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

In various embodiments, the system includes ribs or arms for manipulating clot near the aspiration lumen inlet.

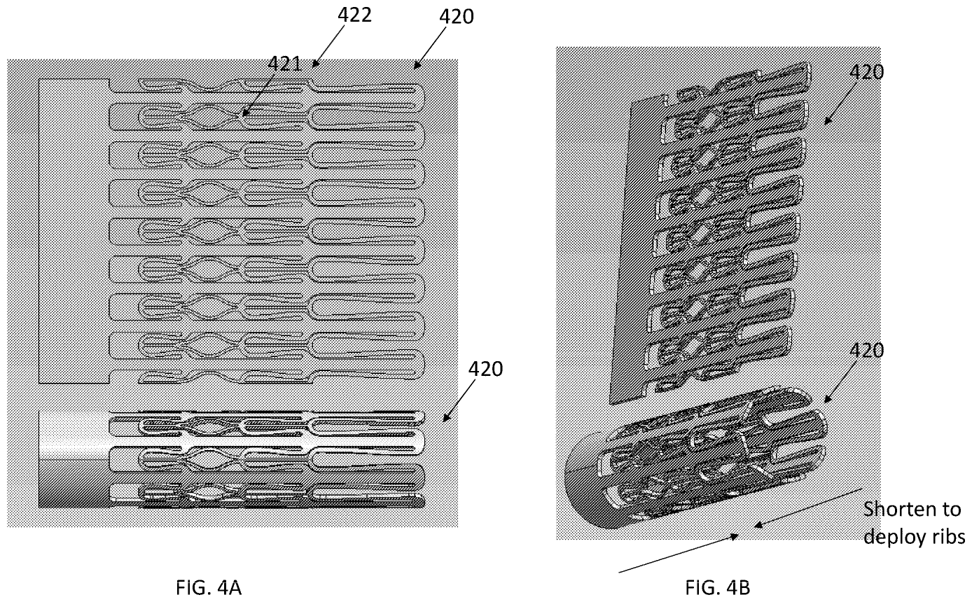

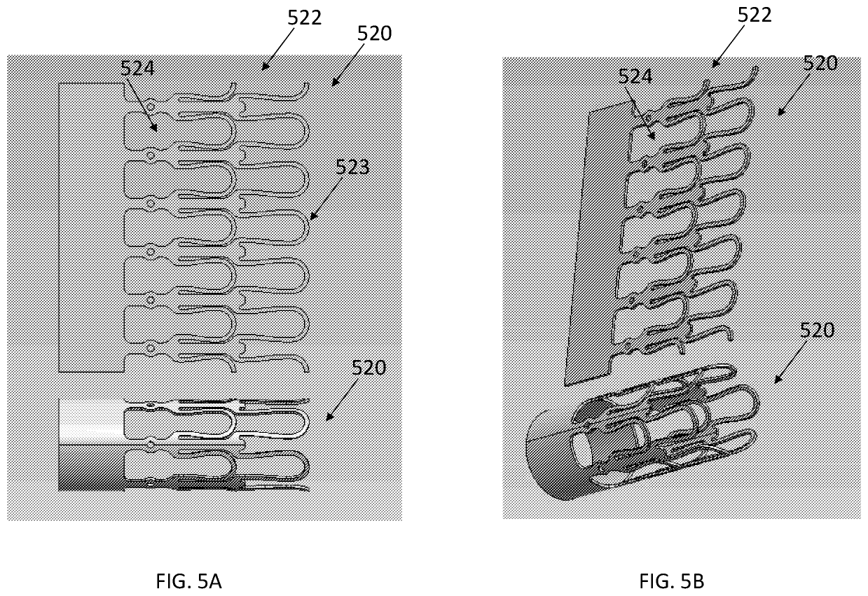

FIGS. 4A and 4B illustrate flattened and constructed views of an exemplary funnel assembly 420 for use in connection with the system described herein. The embodiment of FIGS. 4A-4B has ribs 421 and a frame 422 integrated into a unitary manufactured element. The exemplary cut patterns include integrated ribs 421 which can be deployed when the frame is manipulated (e.g. the distal end of the frame 422 is pulled proximally while the proximal end remains in a fixed position, in other words when the frame is shortened).

The exemplary system can be configured to be a sheathless design. An actuation mechanism expands and retracts the funnel. Although not shown, suitable mechanisms include, but are not limited to, a leaf spring to inflate/deflate the base of the funnel. The ribs can be used, at least in part, to expand/compress the funnel slightly to allow for a guidewire to be passed through easily. In various embodiments, the ribs and funnel frame portions of the material have different transition temperatures (Af temperature) such that they can be actuated independently. In various embodiments, the ribs have a lower Af temperature so they are actuated before the funnel during heating. In various embodiments, the rib structure and frame are thermally separate. For example, the ribs may be insulated from the funnel frame.

Various other configurations may be employed as would be understood by one of skill from the description herein. For example, the catheter system may include inner and outer frame elements. The inner structure may be formed as a frame for the funnel. The outer structure may be formed as an integrated rib structure and funnel frame. Other nested frame designs are contemplated. In various embodiments, the outside frame with ribs can be attached (e.g. riveted) to a section of the funnel frame. In the unactuated state, the ribs would be straight and flush with the funnel walls. With heat applied in the actuated state, the ribs change to a more concave geometry which would pull back on the funnel, foreshorten it's height, and increase the diameter of the distal tip. This may be used to turn the funnel into a wall scraper for adhered clot cases depending on the application.

The system may include other modifications to suit the requirements of different clinical applications. For example the end may be configured so it can be manipulated towards a clock. The funeral may be directed or turned towards a clot or vessel wall. The diameter and shape of the funnel may be manipulated as would be understood by one of skill from the description herein. The dimensions of the system may be modified depending on the vessels in which the system is intended for use or the clinical application. For example, the clinical needs are different for pulmonary embolism, deep veins, arterial vessels, and neurovascular vessels.

### Mechanical Manipulation Features for Engagement With Tissue or Material at Distal End of Device (e.g., Grabber Arms)

Some embodiments of a thrombus removal device can include features enabling mechanical manipulation or engagement with tissue or material at a distal end of the device. Additional details on mechanical manipulation features are described in PCT/US2023/062002, filed Feb. 3, 2023, which is incorporated herein by reference in its entirety. These features can be referred to herein as mechanical engagement features, grabber arms, fangs, mechanical manipulation arms, mechanical cutting arms, or the like. The grabber arms are generally designed and configured to engage and pull clots into the distal end of the device (e.g., the funnel and/or aspiration lumen). In various implementations, the mechanical engagement features disclosed herein are configured to achieve some combination of pulling thrombus into the funnel, pulling thrombus into the jet plane, pulling thrombus into the aspiration lumen, and/or breaking off or cutting pieces of the thrombus into pieces sufficiently small to be aspirated through the aspiration lumen. While the thrombus removal systems described herein generally include an aspiration lumen and one or more fluid streams or jets, it should be understood that the grabber arms may be implemented in devices without aspiration lumens or without one or more fluid streams or jets. Additionally, the devices described herein generally include an expandable funnel on the distal end of the device. However it should be understood that some embodiments with mechanical engagement features may not include an expandable funnel, but instead some other structure on or near the distal end of the device.

Mechanical engagement features can comprise an arrangement of fangs, arms, or actuatable members positionable at a distal end of a device, such as a thrombus removal device. In some embodiments, the mechanical engagement features are positioned along or near an outside surface of the distal end (e.g., funnel). In some aspects, the entirety of the mechanical engagement features are positioned outside of the funnel. In some embodiments, the mechanical engagement features are configured to pass through the funnel or distal end when actuated to enter or be positioned within the funnel or distal end. In some aspects, the distal tip of the mechanical engagement features are configured to pass through the funnel or distal end when actuated to enter or be positioned within the funnel or distal end interior.

The mechanical engagement features described herein do not include any components that extend distally beyond the distal end of the expandable member or funnel. In general, all actuation or movement of the mechanical engagement features is provided within the confines of the expandable member or funnel once the features have passed through/into the funnel. In some embodiments, the mechanical engagement features can include cutting or serrated edges, sharp points, or shearing/pinching mechanisms of action against a targeted clot or tissue. Maintaining the entirety of the mechanical engagement features within the funnel or expandable member increases patient safety and prevents accidentally damaging, cutting, or piercing sensitive tissues such as vessel walls.

The mechanical engagement features described herein can generally include an at-rest state in which the mechanical engagement features are generally not obstructing a central or aspiration lumen of the device (e.g., resting near, adjacent to, or against an outer wall of the expandable member or funnel). The mechanical engagement features can also include an actuated or closed state in which the mechanical engagement features are manipulated to move, either axially and/or radially, through the funnel (e.g., through a frame and/or compliant material of the funnel) towards the central or aspiration lumen of the device. In some embodiments, this manipulation causes the mechanical engagement features to move axially towards the central or aspiration lumen, and in other embodiments, the manipulation causes the mechanical engagement features to move radially through and across the expandable member or funnel towards or across a central axis of the opening.

Generally, actuation or manipulation of the mechanical engagement features results in movement of a portion of the mechanical engagement features, such as at least the distal tips or distal ends of the mechanical engagement features, along a pivot through and within the expandable member or funnel. The pivot provides an inflection point between the mechanical engagement feature and the actuating member (e.g., a pull wire, an outer sheath, etc.). While this disclosure discussed movement of the mechanical engagement features as either being axially (e.g., distal to proximal) or radially (e.g., through and across the funnel or expandable member) it should be understood that since the mechanical engagement features of this disclosure typically move along a pivot, the movement characteristics may be more complex (e.g., a mechanical engagement feature may first swing radially to pass through the funnel towards a center of the expandable member or funnel before then swinging more axially towards the opening or aspiration lumen of the device within the funnel).

The mechanical engagement features described herein typically also are directed or face inwards towards a central axis of the device (as opposed to facing outwards towards a vessel wall.

FIGS. 5A-5B illustrate a top-down view and a side-view, respectively, of one embodiment of a distal end or funnel 520 of a device which can include additional functionality for delivery and therapy. In the illustrated embodiment, the distal end or funnel 520 can include a frame 522 comprising a plurality of petals 523. In some aspects, the frame can comprise a plurality of openings 524. In the illustrated embodiment, the distal end frame is shown having a total of 6 rows and 2 columns of petals, but it should be understood that in other embodiments any number of rows and columns of petals can be implemented, including 2, 3, 4, 5, 6, 7, 8, 9, 10 or more rows or columns of petals and any combination thereof. Also, while the petals and petal features are described independently herein, it should be understood that in some embodiments, the frame is a unitary design in which the entire structure, including the plurality of petals, is typically a unitary structure and is manufactured from a single piece of metal (e.g., the entire pattern is laser cut from a piece of nitinol or other appropriate metal or material). The structure of the distal end frame after cutting and shaping can be a single piece of material (e.g., nitinol).

The openings 524 in the funnel are configured to receive or accommodate one or more mechanical engagement features that are positioned near or adjacent to an exterior surface of the funnel 520. When the mechanical engagement features are actuated, at least a portion of the engagement features that includes the distal tip or distal end is are configured to pass through the openings 524 of the funnel to enter the interior of the funnel. The mechanical engagement features can be generally inwards facing (e.g., facing inwards towards a central axis of the device). As will be described in more detail below, the mechanical engagement features can be manipulated or actuated so as to cause distal tips of the mechanical engagement features 2206 to move or pivot axially towards or away from an aspiration lumen of the device that is in fluid communication with the funnel.

The funnel 520 including the funnel frame 522 can be coated, covered, or encapsulated with a compliant material, such as a urethane or polyurethane material such as Chronoflex. The compliant material can provide an atraumatic and compliant distal end for the thrombectomy device.

In some implementations, the mechanical engagement features can be manipulated or actuated manually by a user of the device, such as by engaging with pull wires or sliding/rotating an outer sheath over the device. In other embodiments, the actuation or manipulation can be automated such as by coupling the engagement features to motors configured to actuate pull wires or translate/rotate an outer sheath. The motors can be controlled by the user, such as by interacting with a user input device on the device handle or console (e.g., buttons, levers, switches, triggers, etc.).

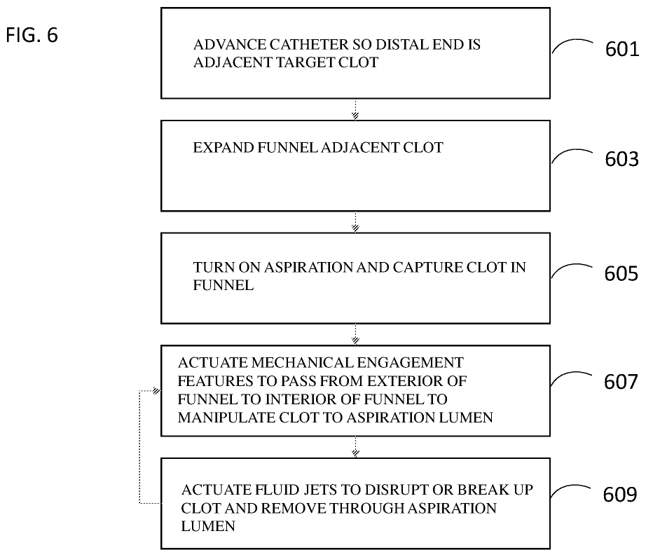

FIG. 6 is a flowchart describing a method of removing a thrombus from a patient. The method of FIG. 6 can include the steps of: step 601, advancing a thrombectomy catheter so a distal end is adjacent to a target clot; step 603, expanding a funnel of the thrombectomy catheter adjacent to the clot; step 605, turning on aspiration in the thrombectomy catheter and capturing the clot in the funnel; step 607, actuating mechanical engagement features to pass at least a portion of the mechanical engagement feature (e.g., at least the distal tips or distal ends) from an exterior of the funnel to an interior of the funnel to manipulate clot to the aspiration lumen; and step 609, actuating fluid jets to disrupt or break up clot and remove through the aspiration lumen. It should be noted that during a procedure, any of the steps 601 through 609 can be repeated or performed in a different order. For example, in some embodiments, the steps of actuating the mechanical engagement features and actuating the fluid jets can be continuously or periodically repeated until the clot is broken up and removed from the patient.

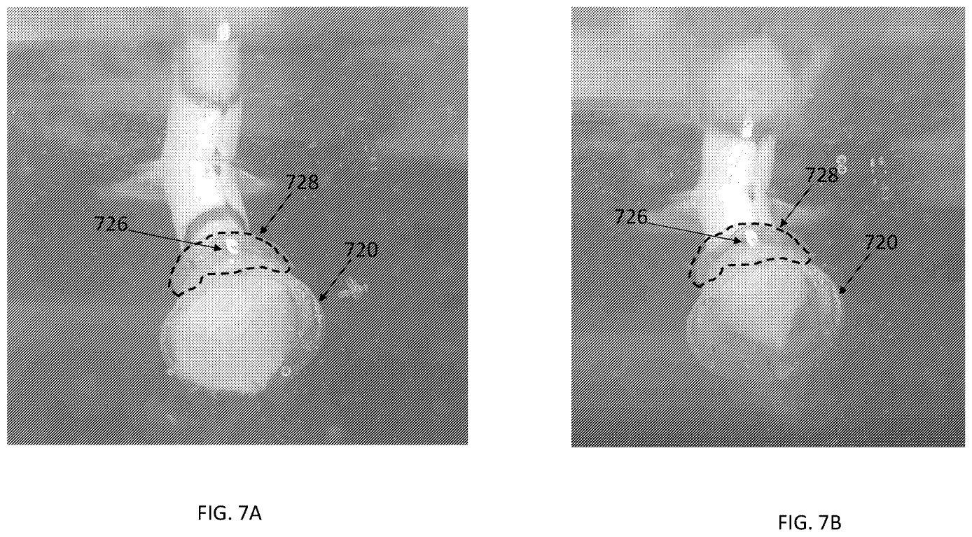

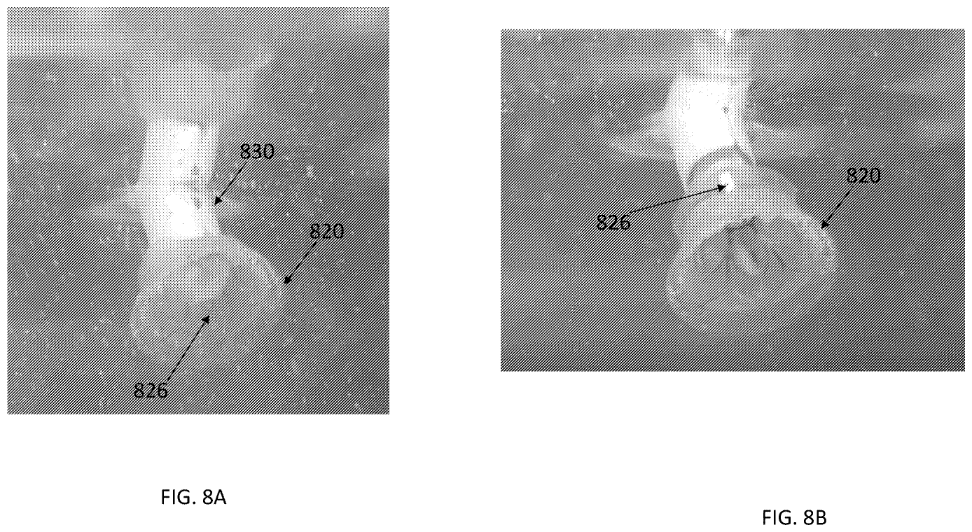

FIGS. 7A, 7B, 8A, and 8B show an exemplary catheter as described above with an expandable funnel in fluid jets.

In FIG. 7A, the funnel is expanded and captures clot. As shown in FIG. 7A, one or more mechanical engagement features 726 are shown positioned at, near, or adjacent to an exterior surface of the funnel. The funnel is shown covered or encapsulated in a compliant material. The one or more mechanical engagement features can further be covered or protected with a second compliant material 728. In some examples, the second compliant material 728 is separate from the compliant material that encapsulates the funnel. In other embodiments, the second compliant material is integral to the funnel compliant material and forms a pocked on the exterior of the funnel frame to accommodate the mechanical engagement features when they are positioned outside of the funnel. FIG. 7B shows the clot being aspirated into the funnel and aspiration lumen, with the mechanical engagement features still positioned exterior to the funnel.

In FIG. 8A, the mechanical engagement features 826 are shown being actuated to pass through openings of the funnel frame into an interior of the funnel 820 to engage with the clot in the funnel. In this example, the mechanical engagement features are actuated by advancing an outer sheath 830 of the device over a portion of the mechanical engagement features to actuate them into the funnel. However, as described herein, other techniques for actuating the mechanical engagement features can be provided. FIG. 8B shows the mechanical engagement features returned to the exterior of the funnel 820 after the clot has been cleared by the funnel and aspiration lumen.

In any of the embodiments described herein, fluid jets of the thrombectomy device can be turned on to break up the clot so it can be adequately aspirated through the catheter without clogging.

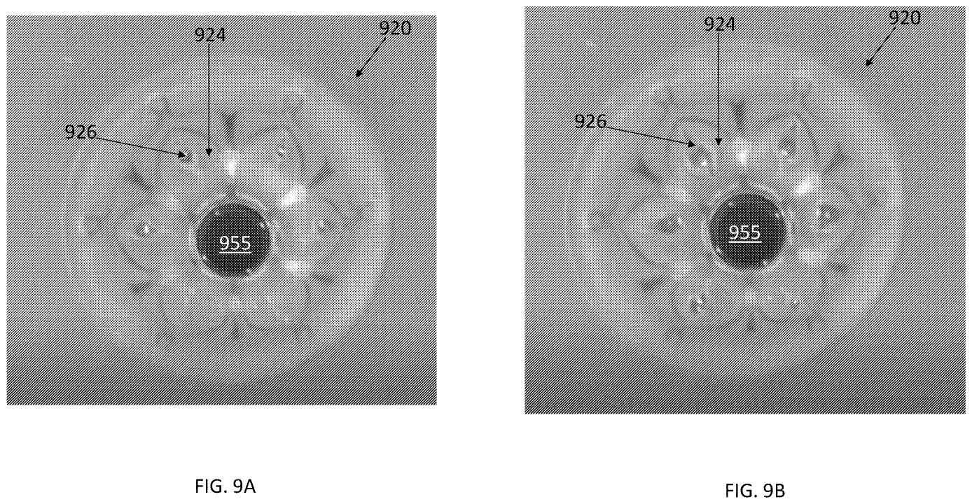

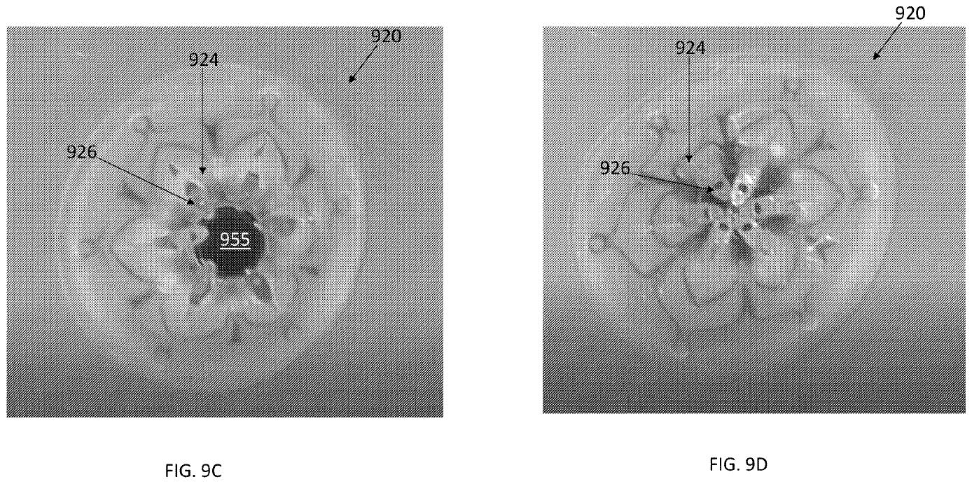

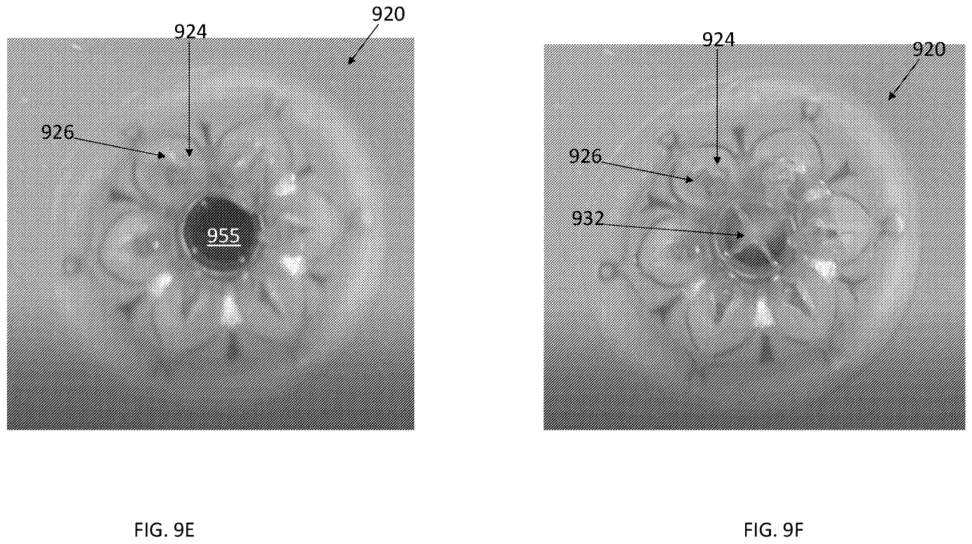

FIGS. 9A-9F is a sequence of figures showing a similar catheter but including mechanical engagement features configured to pass from an exterior of the funnel into an interior of the funnel to manipulate clot material inside the funnel. The catheter is directed towards the target clots so the distal end is adjacent to the clot. The funnel is expanded and aspiration is turned on to capture clot in the funnel, as described above.

In FIGS. 9A to 9D, the mechanical engagement features 926 are actuated to pass from an exterior of the funnel 920 to an interior of the funnel through openings 924 in the funnel frame. The sequence of figures shows the mechanical engagement features progressively getting closer to the aspiration lumen 955 of the device. The compliant material of the frame can also include cuts or slits to allow the mechanical engagement features to pass, or alternatively, the mechanical engagement features can cut or produce the slits in the compliant material of the funnel when actuated. As shown in FIG. 9D, the mechanical engagement features 926 close within the funnel such that any material that was within the funnel is urged towards the mouth of the funnel (e.g., the aspiration lumen 955). In some embodiments, at the same time, fluid jets can be operated to break up the clot material as it enters the aspiration lumen. Operation of fluid jets 932 is shown in FIG. 9F. FIG. 9E shows the mechanical engagement features slightly retracted from the aspiration lumen 955 after the complete actuation shown in FIG. 9D.

The fluid jets may be operated and configured in various manner as described above. For example, the fluid jets may be operated continuously or intermittently. The fluid jets may be operated differently at different phases of the procedure. In an exemplary embodiment, the fluid jets are turned on after clot is captured in the funnel. In an exemplary embodiment, the fluid jets are operated in a pulsating fashion. The jets may be alternated between on and off states (within the pulses) in any of the embodiments described herein.

The jets may be coordinated with the mechanical engagement features. For example, the jets may be turned off when the mechanical engagement features are in an open state and turned on when the mechanical engagement features are actuated towards the aspiration lumen.

In various embodiments, the mechanical engagement features and/or the jets may be operated autonomously. For example, the jets and/or mechanical engagement features may be operated according to a predetermined program. In one example, the jets and/or mechanical engagement features cycle between on/off or open/closed, as the case may be, according to a set frequency.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal device, comprising:
an elongate catheter having an aspiration lumen;
an aspiration source coupled to the aspiration lumen;
an expandable funnel coupled to the aspiration lumen and the elongate catheter;
at least one mechanical engagement feature disposed entirely external to the expandable funnel, wherein the at least one mechanical engagement feature is actuatable to pass through an opening in expandable funnel into an interior of the expandable funnel.

2. The thrombus removal device of claim 1, wherein the at least one mechanical engagement feature is actuatable to move the at least one mechanical engagement towards the aspiration lumen.

3. The thrombus removal device of claim 1, wherein the at least one mechanical engagement feature is actuatable to move the at least one mechanical engagement feature generally radially within the expandable funnel across at least a portion of the expandable funnel.

4. The thrombus removal device of claim 1, wherein the at least one mechanical engagement feature comprises a cutting portion.

5. The thrombus removal device of claim 1, wherein the at least one mechanical engagement feature comprises a blunted tip.

6. The thrombus removal device of claim 1, further comprising:
a fluid source;
a fluid lumen positioned in the elongate catheter and in fluid communication with the fluid source;
a jet orifice positioned near or within the expandable funnel and in fluid communication with the fluid lumen, the jet orifice being configured to provide a fluid stream within the aspiration lumen or within the expandable funnel.

7. The thrombus removal device of claim 6, wherein the at least one mechanical engagement feature is actuatable to move the at least one mechanical engagement within the expandable funnel towards the fluid stream.

8. The thrombus removal device of claim 6, wherein at least one mechanical engagement feature is in fluid communication with the fluid lumen and is further configured to provide a second fluid stream within the aspiration lumen or within the expandable funnel.

9. The thrombus removal device of claim 1, wherein the expandable funnel comprises a funnel frame configured to self-expand the funnel to a fully expanded configuration.

10. The thrombus removal device of claim 9, wherein the expandable funnel further comprises a compliant material disposed over at least a portion of the funnel frame.

11. The thrombus removal device of claim 10, wherein the at least one mechanical engagement feature includes an at-rest configuration in which the at least one mechanical engagement feature is positioned adjacent to or abutting against an exterior surface of the compliant material.

12. The thrombus removal device of claim 10, wherein the compliant material further comprises at least one opening corresponding to each of the at least one mechanical engagement features, wherein the at least one mechanical engagement is configured to pass through the at least one opening in the compliant material when actuated.

13. The thrombus removal device of claim 1, wherein actuation of the at least one mechanical engagement feature causes the at least one mechanical engagement feature to pivot within the expandable funnel.

14. The thrombus removal device of claim 1, wherein the at least one mechanical feature does not extend beyond a distal end of the expandable funnel.

15. The thrombus removal device of claim 1, wherein at least a distal end of the at least one mechanical engagement feature is actuatable to pass through the opening in expandable funnel into the interior of the expandable funnel.

16. A medical device, comprising:
an elongate catheter;
an expandable member positioned at a distal end of the elongate catheter;
at least one mechanical engagement feature disposed exterior to the expandable member, wherein at least a distal end of the at least one mechanical engagement feature is actuatable to pass through an opening in the expandable member into an interior of the expandable member.

17. The medical device of claim 16, wherein the at least one mechanical engagement feature is actuatable to move the at least one mechanical engagement within the expandable member.

18. The medical device of claim 16, wherein the at least one mechanical engagement feature is actuatable to move the at least one mechanical engagement feature generally radially within the expandable member across at least a portion of the expandable member.

19. The medical device of claim 16, wherein the at least one mechanical engagement feature comprises a cutting portion.

20. The medical device of claim 16, wherein the at least one mechanical engagement feature comprises a blunted tip.

21. The medical device of claim 16, further comprising:
a fluid source;
a fluid lumen positioned in the elongate catheter and in fluid communication with the fluid source;
a jet orifice positioned near or within the expandable member and in fluid communication with the fluid lumen, the jet orifice being configured to provide a fluid stream within the expandable member.

22. The medical device of claim 21, wherein the at least one mechanical engagement feature is actuatable to move the at least one mechanical engagement within the expandable member towards the fluid stream.

23. The medical device of claim 21, wherein at least one mechanical engagement feature is in fluid communication with the fluid lumen and is further configured to provide a second fluid stream within the aspiration lumen or within the expandable member.

24. The medical device of claim 16, wherein the expandable member comprises a frame configured to self-expand the expandable member to a fully expanded configuration.

25. The medical device of claim 24, wherein the expandable member further comprises a compliant material disposed over at least a portion of the frame.

26-38. (canceled)
